(19) United States
(12) Patent Application Publication
(10) Pub. No.: US 2012/0059163 A1
(43) Pub. Date: Mar. 8, 2012

(54) Non-linear opticaly active molecules, their synthesis, and use

(75) Inventors: Vincent D. McGinniss, Sunbury, OH (US); Steven M. Risser, Reynoldsburg, OH (US); Elizabeth Drotleff, Worthington, OH (US); Edward Jiang, Columbus, OH (US); Kevin B. Spahr, Worthington, OH (US)

(73) Assignee: Optimer Photonics, Inc., Columbus, OH (US)

(21) Appl. No.: 13/290,518

(22) Filed: Nov. 7, 2011

(30) Foreign Application Priority Data
Apr. 28, 2005 (US) PCT/US05/14418

(51) Int. Cl. C07D 417/04; C07D 261/12; C07D 231/44; C07C 15/12; C07C 255/42; C07C 229/44; C07C 211/45; C07C 15/18; C07D 239/60; C07D 231/08

(52) U.S. Cl. 544/60; 544/303; 548/243; 548/368.1; 548/371.1; 558/403; 560/19; 564/442; 585/25

(57) ABSTRACT

In one aspect, the present invention provides a hyperpolarizable organic chromophore. The chromophore is a nonlinear optically active compound that includes a π-donor conjugated to a π-acceptor through a π-electron conjugated bridge. In other aspects of the invention, donor structures and acceptor structures are provided. In another aspect of the invention, a chromophore-containing polymer is provided. In one embodiment, the chromophore is physically incorporated into the polymer to provide a composite. In another embodiment, the chromophore is covalently bonded to the polymer, either as a side chain polymer or through crosslinking into the polymer. In other aspects, the present invention also provides a method for making the chromophore, a method for making the chromophore-containing polymer, and methods for using the chromophore and chromophore-containing polymer.

### FIELD OF THE INVENTION

The present invention relates in general to nonlinear optically active molecules and, more particularly to hyperpolarizable organic chromophores having useful electro-optical coefficients.

### BACKGROUND OF THE INVENTION

Electro-optic materials alter their physical properties in the presence of an electric field. Typically, when the material is subjected to an electric field, its polarization changes dramatically, resulting in an increase in the index of refraction of the material and an accompanying decrease in the velocity of light through the material. This electric field-dependent index of refraction can be used to encode electric signals onto optical signals. Uses include, for example, switching optical signals and steering light beams.

Many types of electro-optic materials have been utilized for use in electro-optic devices. Among these materials are, inter alia, inorganic materials such as lithium niobate, semiconductor materials such as gallium arsenide, organic crystalline materials, and electrically poled polymer films that include organic chromophores. A review of nonlinear optical materials is provided in L. Dalton, “Nonlinear Optical Materials”, Kirk-Othmer Encyclopedia of Chemical Technology, 4^{th} Edition, Volume 17 (John Wiley & Sons, New York, 1995), pp. 288-302.

Electro-optic poled polymers have many advantages as modulating materials. Their advantages include their applicability to thin-film waveguiding structures, which are relatively easily fabricated and compatible with existing microelectronic processing. These polymers incorporate organic nonlinear optically active molecules to effect modulation. Because organic materials have low dielectric constants and satisfy the condition that n²=∈, where n is the index of refraction and ∈ is the dielectric constant, organic electro-optic will have wide bandwidths. The dielectric constant of these materials (∈=2.5-4) relatively closely matches the propagating electrical and optical waves, which provides for a bandwidth greater than 100 GHz.

In contrast to inorganic materials in which polar optical lattice vibrations diminish their effectiveness, the optical properties of organic nonlinear optical materials depend primarily on the hyperpolarizability of their electrons without a significant adverse contribution from the lattice polarizability Thus, organic nonlinear optical materials offer advantages for ultrafast electro-optic modulation and switching.

For an organic chromophore to be suitable for electro-optic applications, the chromophore should have a large molecular optical nonlinearity, referred to as the first hyperpolarizability (β), and a large dipole moment (μ). A common figure of merit used to compare materials is the value μβ/MW, where MW is the molecular weight of the chromophore. However, materials characterized as having such large μβ/MW values commonly suffer from large intermolecular electrostatic interactions that lead to intermolecular aggregation resulting in light scattering, unacceptably high values of optical loss, and low EO values. Many of these difficulties can be attributed to the electron acceptor, which induces a large molecular dipole moment, leading to the problems associated with chromophore aggregation.

Thus, the effectiveness of organic nonlinear optical materials having high hyperpolarizability and large dipole moments is limited by the tendency of these materials to aggregate when processed into polymers with low refractive index. Accordingly, there exists a need for improved nonlinear optically active materials having large hyperpolarizabilities and large dipole moments and that, when employed in electro-optic devices, exhibit large electro-optic coefficients. The present invention seeks to fulfill these needs and provides further related advantages.

### SUMMARY OF THE INVENTION

In one aspect, the present invention provides a hyperpolarizable organic chromophore. The chromophore is a nonlinear optically active compound that includes a π-donor conjugated to a 7-acceptor through aπ-electron conjugated bridge. In other aspects of the invention, donor structures and acceptor structures are provided.

In another aspect of the invention, a chromophore-containing polymer is provided. In one embodiment, the chromophore is physically incorporated into the polymer to provide a composite. In another embodiment, the chromophore is covalently bonded to the polymer, either as a side chain polymer or through crosslinking into the polymer.

In other aspects, the present invention also provides a method for making the chromophore, a method for making the chromophore-containing polymer, and methods for using the chromophore and chromophore-containing polymer.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 shows the absorption spectrum of a chromophore of this invention absorbed into a commercial highly fluorinated polymer.

The drawings will be described in more detail below.

### DETAILED DESCRIPTION OF THE INVENTION

In one aspect, the present invention provides a hyperpolarizable organic chromophore. The chromophore is a nonlinear optically active compound that includes a π-donor conjugated to a π-acceptor through a π-electron conjugated bridge.

The chromophores of the invention are characterized as having high electro-optic coefficients; large hyperpolarizability; large dipole moments; chemical, thermal, electrochemical, and photochemical stability; low absorption at operating wavelengths (e.g., 1.3 and 1.55 μm); suitable solubility in solvents suitable for the host polymers detailed below; compatibility with host polymer; and low volatility.

Absorption Maximum (λₘₐₓ).

The nonlinear optical properties of chromophores are strongly correlated with the absorption maximum of the chromophore. As λₘₐₓ increases the NLO properties increase. The absorption maximum of the chromophores have been both computationally determined and experimentally measured.

### Dipole Moment and Optical Hyperpolarizability (β)

Nonlinear optical effects of organic materials depend mainly on the compound's hyperpolarizability (β) and the magnitude of its intrinsic dipole moment (μ). The molecular first hyperpolarizabilities (β) were calculated from the experimentally determined absorption maximum using the expression log (β)=3.6118 log (λₘₐₓ)−7.8324 (Harper). The dipole moments were calculated using ab initio electronic structure methods as implemented in JAGUAR™ (Jaguar 4.0, Schrodinger Inc., Portland, Oreg., 1991-2000). Ab initio methods have been shown to provide accurate descriptions of the dipole moments in organic molecules. All the chromophore geometries were optimized using DFT methods with the B3LYP functional and the 6/31G* basis set. These resulting geometries were used for the calculation of the dipole moments.

### Chromophore Figure-of-Merit (FOM)

The chromophore figure-of-merit (FOM) is determined by the formula FOM=μβ/(molecular weight). This is an approximate measure of how good a chromophore is. Ideally, a chromophore would have a high FOM, while being soluble in the polymers of interest. In reality, increasing the chromophore FOM typically reduces the chromophore solubility and increases its tendency to aggregate, reducing the measurable EO response of the material. The best chromophores are those that have large FOM values while still remaining active in the polymers of interest.

### Chromophore Solubility/Aggregation

Intermolecular attractive forces and limited chromophore solubility in the polymer matrix can cause chromophore aggregation, leading to diminished electro-optic coefficient. Improving chromophore design can reduce/eliminate aggregation by increasing chromophore solubility and reducing chromophore aggregation, leading to enhanced electro-optic coefficients.

Many molecules can be prepared having high hyperpolarizability values, however their utility in electro-optic devices often is diminished by the inability to incorporate these molecules into a host material with sufficient non-centrosymmetric molecular alignment to provide a device with acceptable electro-optic activity. Molecules with high hyperpolarizability typically exhibit strong dipole-dipole interactions in solution or other host material that makes it difficult, if not impossible, to achieve a high degree of non-centrosymmetric order unless undesirable spatially anisotropic intermolecular electrostatic interactions are minimized. In addition, molecules with high hyperpolarizability commonly have limited solubility in low refractive index polymers, which also leads to the aggregation and the reduction of the electro-optic activity.

In certain embodiments, the chromophores of the invention include substituents that enhance the solubility of the chromophore in low refractive index polymers. In one embodiment, the chromophore includes one or more substituents on the donor group portion of the chromophore. In another embodiment, the chromophore includes one or more substituents on the bridge portion of the chromophore. In a further embodiment, the chromophore includes one or more substituents on the acceptor portion of the chromophore. The chromophores of the invention can include combinations of donors, bridges, and acceptors, one or more of which can include substituents to enhance solubility. Thus, in certain embodiments, the invention provides chromophores having one or more substituents effective to reduce chromophore aggregation. In certain embodiments, the invention also provides for substituents on the donor, or bridge, or acceptor, which alter the chromophore shape that reduce the disadvantageous molecular interactions by sterically inhibiting the close approach of chromophores. In these embodiments, the active portion of the chromophore is embedded within the molecular structure that effectively insulates the chromophore dipole from interaction with other such dipoles. Thus, through the use of substitution and control of chromophore shape, the chromophores of the invention provide high electro-optic coefficients when incorporated into electro-optic devices

### Film Quality

Films of the chromophores and polymer, at the specified chromophore weight percentage, were cast onto quartz blanks from dioxane solution. After drying, the films were judged for their optical clarity and homogeneity on a scale of 1-10, with 1 being a completely perfect film. Polymer/chromophore solutions were deemed to be of sufficient quality for further testing if the film scored higher than a 4 on this scale. A set of reference films was employed to ensure consistency in the film evaluation.

### Refractive Index Measurement

For chromophore/polymer films of sufficient optical quality, the refractive indices of the materials will be measured at 1550 nm using the prism coupling method [H. Onodera, I. Awai, and J.-I. Ikenoue, “Refractive index measurement of bulk materials: prism coupling method”, Applied Optics, Vol. 22, 1194-1197, (1983)]. In this method, a thin film is formed on a prism face, and by measuring the power as a function of incidence angle for a 1550 nm laser beam, the refractive index of the film is measured.

Electro-Optic Coefficient (r₃₃)

A chromophore's electro-optic coefficient (r₃₃) can be measured in a polymer matrix using a transmission technique at telecommunication wavelength of 1.55 μm. A representative method for measuring the electro-optic coefficient is described in Nahata, et al., “Electro-optic Determination of the Nonlinear-Optical Properties of a Covalently Functionalized Disperse Red 1 Copolymer”, J. Opt. Soc. Am. B, Vol. 10, pp. 1553-1564 (1993).

Ideally, the electrooptic coefficient of a material is determined solely from the FOM of the chromophore and the loading of the chromophore in the host polymer. Thus the largest response would be obtained by placing as much of a high FOM chromophore as is possible within any refractive index constraints there may be. In reality, chromophores with higher FOM values have a stronger tendency to perform non-ideally. This invention, then, is concerned with chromophores that possess large FOM values, but also exhibit large electrooptic coefficients when placed in the polymers of interest.

The chromophores of the invention are characterized as having an electrooptic coefficient (r₃₃) of at least about 5 pm/V at 100V/m poling voltage measured at 1.55 μm in a fluoropolymer with a compound loading of about 5% to 20% by weight based on the total weight of fluoropolymer. The chromophore loading is determined by ensuring the refractive index of the composite system is approximately 1.475 or less at room temperature. This refractive index ensures the material can be used as cladding on silica waveguides when at elevated temperature. When there is no need to maintain low refractive index, the chromophores of this invention may also be incorporated into conventional polymers at higher concentrations, leading to correspondingly higher electrooptic coefficients.

### Representative Chromophore Components and Structures

In addition to providing chromophores, the present invention provides donors, bridges, and acceptor components useful in the construction of chromophores for electro-optic device adaptation. As noted above, the chromophores of the invention include: (1) a π-donor conjugated to (2) a π-acceptor through (3) aπ-electron conjugated bridge.

### Donors

Representative donor structures from the prior art are illustrated below.

The donor typically consists of an aromatic ring with an attached amino group. Other donors include, for example, cyclohexene, furan, or thiophene rings with an attached amino group.

In certain embodiments, the donor includes a bulky substituent to enhance chromophore solubility or inhibit chromophore aggregation. In other embodiments, the donor includes a functional group (e.g., trifluorovinyl ether) suitable for attachment as a polymer side chain, or for crosslinking to either a polymer matrix or other suitably functionalized chromophores. Inclusion of the bulky substituent or functional group tends to decrease the figure of merit for the chromophore, as the molecular weight is increased while the nonlinearity and dipole moment tend to remain unchanged. This decrease in the chromophore figure of merit can often be partially offset by including more chromophore in the chromophore/polymer blend, while remaining within whatever refractive index constraints may exist for the total material. [L. Dalton, Nonlinear Optical Polymeric Materials: From Chromophore Design to Commercial Applications, “Advances in Polymer Science” vol. 158, K.-S. Lee Editor, Polymers for Photonic Applications I, pages 1-86, Springer, New York, 2002]

### Bridges

Representative bridge structures from the prior art are illustrated below. The bridge structure is a π-electron conjugated bridge. The bridge can include a variety of groups including, for example, a dihydrofuran group, a fused dithiophene group, a fused trithiophene group, a dithiophene group, and substituted versions of these groups.

[H. Singh Nalwa et. al., Organic Materials for Second-Order Nonlinear Optics in “Nonlinear Optics of Organic Molecules and Polymers”, edited by H. S. Singh Nalwa and S. Miyata, Chapter 4, pages 89-350, CRC Press, Boca Raton, Fla., 1997]
[A. Harper et al, J. Opt. Soc. Am. B, vol. 15, pages 329-339 (1998)]
[L. R. Dalton et al, J. Mater. Chem., vol. 9, pages 1905-1920 (1999)

In certain embodiments, the π-electron conjugated bridge includes a bulky substituent to inhibit chromophore aggregation. Included among these substituents are alkyl substituents.

It will be appreciated that chromophores of the invention can include any combination of donors, bridges, acceptors, substituted donors, substituted bridges, and substituted acceptors, described herein.

As used herein, the term “alkyl group.” refers to branched or straight chain alkyl groups. Alkyl groups include from one to ten or more carbon atoms that are unsubstituted or substituted. The alkyl groups include substituents for coupling to a main chain polymer, substituents for coupling to a crosslinking group, or crosslinking substituents.

The following bridge structures may also be used for the chromophores of this invention.

### Acceptors

Representative prior art acceptor structures are illustrated below. The acceptor consists of multiple electron withdrawing groups, typically cyano groups, joined to a conjugated linker or conjugated fused ring. The addition of the third cyano group greatly reduces the solubility of the chromophores in low-refractive index polymers, and increases the tendency of the chromophores to aggregate in all polymers. The solution in the prior art to the aggregation problem has been to include bulky groups either on the donor or the acceptor itself.

[A. W. Harper, Systematic Optimization of Second Order Optical Nonlinearities in Molecules and Polymers, Ph.D. Dissertation, 1997, Univ. of Southern California]

### Chromophore-Containing Polymers

In another aspect of the invention, chromophore-containing polymers are provided. These polymers include any one of the chromophores described above, including the chromophore-containing macromolecular structures. In one embodiment, the chromophore is physically incorporated into a polymer to provide a composite. In another embodiment, the chromophore is covalently incorporated into the polymer by, for example, attachment as a side chain or crosslinking. In one embodiment, the chromophore is crosslinked to the polymer in more than one position, for example, a double-ended crosslinked chromophore.

Generally, once a chromophore of appropriate optical nonlinearity (pp), optical absorption, and stability has been identified, the material is processed into a polymeric material that contains acentrically-aligned chromophores.

To withstand processing conditions and operational conditions (optical power levels at 1.3 and 1.55 microns), chromophore-containing polymers are hardened subsequent to electric field poling to withstand temperatures of 90° C. or greater. As noted above, in certain embodiments, the chromophores include reactive functional groups (e.g., hydroxyl groups) that permit processing into hardened polymer matrices. When thermosetting chemical reactions are employed to lock-in electric field poling-induced acentric order, a stepped poling procedure can be used in which temperature and electric field strength is increased in successive steps to optimize material electro-optic activity.

The chromophores can be incorporated into a variety of host materials including, for example, poly(methyl methacrylate) (PMMA); or copolymers of methyl methacrylate with trifluoroethyl methacrylate.

In summary, suitable methods for incorporating a chromophore into a polymer include the steps of combining the chromophore with the polymer; electric field poling of the chromophore/polymer mixture to acentrically align chromophores; followed by crosslinking, curing, and hardening the chromophore-containing polymer.

### Electro-optic Devices

Components of optical communication systems that may be fabricated, in whole or part, with materials of the present invention include, without limitation, straight waveguides, bends, single-mode splitters, couplers (including directional couplers, MMI couplers, star couplers), routers, filters (including wavelength filters), switches, modulators (optical and electrooptical, e.g., birefringent modulator, the Mach-Zehnder interferometer, and directional and evanescent coupler), arrays (including long, high-density waveguide arrays), optical interconnects, optochips, single-mode DWDM components, and gratings.

The materials of the present invention may be used in telecommunication, data communication, signal processing, information processing, and radar system devices and thus may be used in communication methods relying, at least in part, on the optical transmission of information. Thus, the present invention provides a method of communication comprising transmitting information by light, the light transmitted at least in part through a material of the present invention.

In various embodiments, the present invention provides one or more of:
An EO device comprising at least one of a chromophore, a composition, or a composition prepared by a process according to the present invention;
A waveguide comprising at least one of a chromophore, a composition, or a composition prepared by a process, according to the present invention;
An optical switch comprising at least one of a chromophore, a composition, or a composition prepared by a process, according to the present invention;
An optical modulator comprising at least one of a chromophore, a composition, or a composition prepared by a process, according to the present invention;
An optical coupler comprising at least one of a chromophore, a composition, or a composition prepared by a process, according to the present invention;
An optical router comprising at least one of a chromophore, a composition, or a composition prepared by a process, according to the present invention;
A communications system comprising at least one of a chromophore, a composition, or a composition prepared by a process, according to the present invention;
A method of data transmission comprising transmitting light through at least one of a chromophore, a composition, or a composition prepared by a process, according to the present invention;
A method of telecommunication comprising transmitting light through at least one of a chromophore, a composition, or a composition prepared by a process, according to the present invention;
A method of transmitting light comprising directing light through or via at least one of a chromophore, a composition, or a composition prepared by a process, according to the present invention;
A method of routing light through an optical system comprising transmitting light through or via at least one of a chromophore, a composition, or a composition prepared by a process, according to the present invention;
An interferometric optical modulator or switch, comprising: 1) an input waveguide; 2) an output waveguide; 3) a first leg having a first end and a second end, the first leg being coupled to the input waveguide at the first end and to the output waveguide at the second end; and 4) and a second leg having a first end and a second end, the second leg being coupled to the input waveguide at the first end and to the output waveguide at the second end, wherein at least one of the first and second legs includes a composition of matter according to the present invention;
An optical modulator or switch, comprising: 1) an input; 2) an output; 3) a first waveguide extending between the input and output; and 4) a second waveguide aligned to the first waveguide and positioned for evanescent coupling to the first waveguide; wherein at least one of the first and second legs includes a composition of matter according to the present invention. The modulator or switch may further including an electrode positioned to produce an electric field across the first or second waveguide; or
An optical router comprising a plurality of switches, wherein each switch includes: 1) an input; 2) an output; 3) a first waveguide extending between the input and output; and 4) a second waveguide aligned to the first waveguide and positioned for evanescent coupling to the first waveguide; wherein at least one of the first and second legs includes a composition of matter according to the present invention. The plurality of switches may optionally be arranged in an array of rows and columns.

The following examples are provided for the purpose of illustrating, not limiting, the invention.

### EXAMPLES

### General Materials and Methods

In the following examples, all analytical grade reagents and solvents were purchased from commercial sources including, inter alia, SigmaAldrich and VWR Scientific Products. The starting materials and solvents were used without further purification unless otherwise noted. When dry reaction conditions are indicated, the solvents used are Aldrich Sure Seal grade; transferred via dry syringe or cannula. All reactions involving moisture or oxygen sensitive materials were performed in flame-dried glassware under a positive pressure of argon. Silica gel used for column chromatography was obtained from Aldrich with a mesh of 70-230 ASTM. Thin layer chromatography was done using EMS Science Silicagel 60 F₂₅₄. plates. All proton and carbon nuclear magnetic resonance spectra were recorded on a Bruker 600 MHz FTNMR spectrometer using CDCl₃ and tetramethylsilane as an internal reference. Visible spectra were obtained using a Shimadzu UV160U, using CHCl₃ as the solvent, and a nominal chromophore concentration of 1×10⁻⁵ on a weight basis.

### Host Polymers

The chromophores of this invention were evaluated in one or more of the following low-refractive index polymers. The polymers were synthesized using standard procedures. Polymer A (SPIKE) is a terpolymer composed of 14.4 mol % trifluoroethyl methacrylate, 80.6 mol % tetrafluoropropyl methacrylate, and 5 mol % hydroxyethyl methacrylate. The refractive index of this polymer at 1550 nm was measured to be n=1.4181. Polymer B (Run 46) is a terpolymer composed of 21 mol % trifluoroethyl methacrylate, 52 mol % tetrafluoropropyl methacrylate, and 27 mol % hydroxyethyl methacrylate. The refractive index of this polymer at 1550 nm was measured to be n=1.4338. Polymer C (CP001) is a terpolymer composed of 21 mol % trifluoroethyl methacrylate, 25 mol % heptafluoroisopropyl methacrylate and 25 mol % hydroxyethyl methacrylate. The refractive index of this polymer at 1550 nm was measured to be n=1.4189. Polymer D (CP062) is a terpolymer composed of 35 mol % trifluoroethyl methacrylate, 60 mol % difluoroethyl methacrylate and 5 mol % hydroxyethyl methacrylate. The refractive index of this polymer at 1550 nm was measured to be n=1.4322. Polymer E (CP051) is a copolymer composed of 50 mol % trifluoroethyl methacrylate, and 50 mol % methyl methacrylate. The refractive index of this polymer at 1550 nm was measured to be n=1.4384. Polymer F (CP088) is a terpolymer composed of 65 mol % tetrafluoropropyl methacrylate, 30 mol % methyl methacrylate, and 5 mol % hydroxyethyl methacrylate. The refractive index of this polymer at 1550 nm was measured to be n=1.4368. Polymer G (CP087) is a copolymer composed of 85 mol % tetrafluoropropyl methacrylate and 15 mol % hydroxyethyl methacrylate. The refractive index of this polymer at 1550 nm was measured to be n=1.4266. Polymer H(CP089) is a copolymer composed of 50 mol % tetrafluoropropyl methacrylate and 50 mol % methyl methacrylate. The refractive index of this polymer at 1550 nm was measured to be n=1.4378. Polymer I (CP090) is a terpolymer composed of 65 mol % tetrafluoropropyl methacrylate, 20 mol % tetrafluorobutyl methacrylate, and 15 mol % hydroxyethyl methacrylate.

Other polymers that can be used in this invention may have either low (n<1.5) or high (n>1.5) refractive index values, and include the following:

Thermoplastic Polymers: fluorinated acrylics, polyesters, polyimides, nylons, polycarbonates, polysulfones, and non-fluorinated nylons, acrylics, polyesters, polyimides, polycarbonates, and polysulfones.

Crosslinked Polymers: acrylics, polyesters, unsaturated polymers, polymers substituted with reactive groups (—COOH, —OH, —NCO, —Si(OCH₃)₃, —OCF═CF₂) polyethers.

Branched polymers, interpenetrating polymer networks, and dendrimers [Bosman et al, Chem. Rev. 99, 1665, (1999)].

Additionally, the chromophores of this invention may be attached as side chain groups to these polymers.

Silicon Polymers: silicon polymers can contain the following backbone structures

where R aliphatic, aromatic, or fluoro-hydrocarbon mixtures.

Examples of commercial silicon polymers and resins can be found in United Chemical Technologies (UCT) Product Catalog 2005 (1-800-541-0559) [www.unitedchem.com]

The chromophores of this invention can be either blended into silicon polymers directly, or can be incorporated by modification of the chromophore for direct reaction into the silicon polymer backbone

The silicon polymers can have refractive index values that range between 1.38 and 1.53.

### General Synthetic Routes for Donors (D) and Bridge (B) Structures (D-B)

### General Synthetic Routes for Donor-Bridge (D-B) Acceptor (A) Structures (D-B-A):

### New Fluorinated Fused Ring Acceptor Structures:

The asterisk denotes the active methylene, and X_{F} is given by

and X_{H} is given by

Also included as novel fluorinated fused ring acceptors are

Synthesis of barbituric and thiobarbituric acid derivatives can be found in J. March, Advanced Organic Chemistry, J. Wiley & Sons, New York, 1985, pp 379, while the pyrazolones are described on pp 804.

### New Fluorinated Fused Ring Acceptor Structures:

Synthesis of these structures is described in Australian Journal of Chemistry, Vol. 52, pp 1029-1033 (1999).

### New Active Amine Acceptor Structures:

The following structures are typical active amine acceptor structures

TABLE 1
Examples of Active Amine Acceptor Structures

X₇
—H

### Modifications of Amine-Functional Active Methylene Acceptor Structures:

The following structures are typical amine-functional active methylene acceptor structures:

The modifications to the above molecular structures that were made as part of this invention were as follows. References for the reactions can be found in J. March, Advanced Organic Chemistry, J. Wiley & Sons, New York, 1985, pp 370, pp 445, and pp 802.

1) First react the active methylene acceptor with a model donor-bridge (D-B) intermediate molecule such as

2) The next reaction sequences for this invention involve the addition of fluorinated alkyl or aryl isocyanates, acid chlorides or sulfonyl chlorides to the amine-functional acceptor-bridge-donor (A-B-D) model compounds previously described. The results of these reactions are shown in the following:

The resulting new acceptor structures can then be characterized as

where R is the ligand resulting from the addition of fluorinated alkyl or aryl isocyanate, acid chloride or sulfonyl chloride to the amine-functional acceptor.

### New Active Methylene Acceptor Structures:

The following new acceptor structures are considered part of this invention.

References for the synthesis of these acceptor structures is J. Fluorine Chemistry, Vol. 66, pp 301-309 (1994) and J. Am. Chem. Soc., Vol. 81, pp 4882-4885 (1959).

TABLE 2
Active Methylene Acceptor Structures.

| X₁ | Y₁ |
|---|---|
| —SO₂CF₃ | |
| —SO₂CF₃ | |
| —SO₂CF₃ | —CN |
| —SO₂CF₃ | |
| | |
| | |
| | |
| | |
| | —CN |
| | —NO₂ |
| | —NO₂ |
| | |
| | |
| | |
| | |
| | |
| | |
| | |
| | |
| | |
| | |
| | —NO₂ |
| —CF₃ | —CHO |
| | X = alkyl, —CH₂(CF₂)ₙCF₃ (n = 0-4) |
| | X = alkyl, —CH₂(CF₂)ₙCF₃ (n = 0-4) |
| | X = alkyl, —CH₂(CF₂)ₙCF₃ (n = 0-4) |
| | X = alkyl, —CH₂(CF₂)ₙCF₃ (n = 0-4) |
| | |
| | |
| | |
| | |

TABLE 3
Active Methylene Acceptor Structures.

| X₂ | Y₂ |
|---|---|
| | |
| | |
| | |
| | |
| | |
| | |
| | |
| | |
| | |
| | |
| | —OX X = alkyl, —CH₂(CF₂)ₙCF₃ (n = 0-4) |
| | —H |
| | —OX X = alkyl, —CH₂(CF₂)ₙCF₃ (n = 0-4) |
| | —H |
| | |
| —CF₃ or —CH₃ | |
| —CF₃ | |

TABLE 4
Active Methylene Acceptor Structures

| X₃ | Y₃ |
|---|---|
| | |
| | |
| | |
| | |
| | |
| | |

TABLE 5
Active Methylene Acceptor Structures

X₄

TABLE 6
Active Methylene Acceptor Structures

X₅

TABLE 7
Active Methylene Acceptor Structures
*CH₃—X₆
X₆

The following is also a new active methylene acceptor structure

### Spatial Donor Structures

Normal EO chromophores can be represented by the D-B-A structural form, or explicitly showing a common donor

Chromophores also can be synthesized according to the following scheme:

Molecular weights of the linear and branched spacers can range from 84 to 1000, and can be aliphatic, aromatic, heteroatomic, partially fluorinated or fully fluorinated structures with refractive indices above or below 1.50.

### New Donor-Bridge Structures

where A is any of the active methylene structures shown above and R₁ and R₂ are prior art donor ligands or donor ligands of this invention, as shown in Example 1.

### New Bridge Structures

The following may be used alone, or in conjunction with other groups to form the bridge structures for the chromophores of this invention (Syn. Comm. 33, 2487-2496 (2003); J. Am. Chem. Soc. 108, 452-461 (1986)).

The general reaction scheme for bridge structures having both ketone and aldehyde functionality is to convert the ketone functionality into the donor portion of the chromophore and convert the aldehyde functionality into the acceptor portion of the chromophore.

where the bridge is selected so as to maintain an aromatic or conjugated pathway between the aldehyde and ketone functionalities.

For bridge structures with dialdehyde functionality, the general reaction scheme is as follows:

where the bridge is selected so as to maintain an aromatic or conjugated pathway between the two aldehyde functionalities.

These novel bridge ketone-functional structures can be reacted with

to form the following novel donor-bridge structures, where R₁ and R₂ are either prior art substituents or the new substituents of this invention.

Prior art donor structures or the donor structures of this invention can also be used with these new bridge structures as can prior art acceptors or the new acceptors of this invention.

Similar reactions can occur with aromatic or aliphatic dialdehydes such as:

In addition to the above structures, the following also form novel bridge structures:

where M=S, O, and R₁ and R₂ are as defined previously.

The following may also be used alone or in conjunction with other groups to form bridge structures (Tetrahedron 54, 2161-2168 (1998); Synthesis, 921-925 (1986); Synthesis, 690-692 (1980); J. Heterocyclic Chem. 13, 253-256 (1976))

With these structures the ketone functionality can be used to attach the acceptor group while the active CH₃ group can be used to create the donor portion of the chromophore

### New Bridge Structures and Arrangement

The following may be used alone, or in conjunction with other groups to form the bridge structures for the chromophores of this invention.

Synthesis of the chromophores of this invention using these bridge structures differs from standard approaches in that the double bond emerging from the ring(s) is oriented in the direction of the donor portion of the chromophore. This chromophore design is also possible with the bridge structures described elsewhere in this invention, and those mentioned in the prior art.

The following examples of chromophores are presented to demonstrate possible donors and acceptors that can be used with these bridge structures.

### Additional Donor-Bridge Structures

The following may be used in conjunction with the acceptors of this invention, or those from the prior art, to form chromophores of this invention.

where M₁=O, S; M₂=O, S, and M₁=, ≠M₂. Z=—H, —O—CH₂—(CF₂)ₙ=₁₋₅—CF₃, —CH₂—(CF₂)ₙ=₁₋₈—CF₃, —(CF₂)ₙ₌₁₋₅—CF₃, and R_{A} independently are any perfluorinated, fluorinated, or non-fluorinated aliphatic or aromatic group with 1-30 carbon atoms functionalized with zero or more of the following functional groups: hydroxyl, ether, ester, amino, silyl, siloxy, or the R_{A} are independently R₁ and R₂ as defined previously.

The characteristics of the chromophore are shown in Table 8 using —CH₂(CN)₂) as the acceptor.

TABLE 8
Properties of Chromophores with Fused Ring Bridge Structures

| M₁ | M₂ | Z | λₘₐₓ | Solubility |
|---|---|---|---|---|
| S | S | H | ~625 nm | poor |
| S | O | H | ~600 nm | intermediate |
| O | S | H | ~600 nm | intermediate |
| O | O | H | ~575 nm | good |

Note:
The absorption maxima are estimated from related compounds and differences in the absorption maxima for the parent donor/bridge compounds.

Thus we see the trade-off between better NLO performance (larger λₘₐₓ) and solubility of the chromophore.

The five-member rings may also be used as part of the donor structure, as in the following

where M₁=O, S; M₂=O, S, and M₁=, ≠M₂. R_{A} independently are any perfluorinated, fluorinated, or non-fluorinated aliphatic or aromatic group with 1-30 carbon atoms functionalized with zero or more of the following functional groups: hydroxyl, ether, ester, amino, silyl, siloxy, or the R_{A} are independently R₁ and R₂ as defined previously.

Additionally, the following may be used as bridge structures; in conjunction with donors, acceptors, and possibly other bridge components of this invention and/or the prior art.

where M=O, S, Z=—O—CH₂—(CF₂)ₙ₌₁₋₅—CF₃, —CH₂—(CF₂)ₙ₌₁₋₈—CF₃, —(CF₂)ₙ₌₁₋₅—CF₃.

The following may be used as bridge/acceptor structures, in conjunction with donors, and possibly other bridge components of this invention and/or the prior art.

where M=O, S. Z=—O—CH₂—(CF₂)_{n=1,5}—CF₃, —CH₂—(CF2)_{n=1,8}—CF₃, —(CF2)_{n=1.5}—CF₃.

Examples of complete chromophores using these components include the following:

where M=O, S, A=—O—CH₂—(CF₂)ₙ₌₁₋₅—CF₃, —CH₂—(CF₂)ₙ₌₁₋₈—CF₃, —(CF₂)ₙ₌₁₋₅—CF₃.

### Example 1

### Chromophores with Novel Donor Structures

In this example, we show the preferred structure of the chromophores of this invention. The general structure of these chromophore is shown below, where B is the π-conjugated or aromatic bridge, and A is the π-acceptor. If R₁ or R₂ contain one or more fluorine atoms, or reactive groups, care must be taken to isolate these groups from the amine to minimize any averse impact on the absorption maximum or dipole moment of the chromophore.

TABLE 9
Possible Donor Structures

| R₁ | R₂ |
|---|---|
| Alkane hydrocarbon CₙH₂ₙ₊₁, n = 1-5 | Alkane hydrocarbon CₙH₂ₙ₊₁, n = 1-5 |
| (Prior art) | (Prior art) |
| | Alkane hydrocarbon CₙH₂ₙ₊₁, n = 1-5 (Prior art) |
| | |
| | Alkane hydrocarbon CₙH₂ₙ₊₁, n = 1-5 (This invention) |
| | |
| | Alkane hydrocarbon CₙH₂ₙ₊₁, n = 1-5 (This invention) |
| | |
| Partially fluorinated alkane hydrocarbon | Alkane hydrocarbon CₙH₂ₙ₊₁, n = 1-5 |
| CₙFₘH₂ₙ₊₁₋ₘ, n = 1-5, m = 1, 2n−1 | (This invention) |
| (This invention) | |
| | Alkane hydrocarbon CₙH₂ₙ₊₁, n = 1-5 (This invention) |
| | Alkane hydrocarbon CₙH₂ₙ₊₁, n = 1-5 (This invention) |
| F₃C—CH₂—O—CH₂— | Alkane hydrocarbon CₙH₂ₙ₊₁, n = 1-5 |
| (This invention) | (This invention) |
| Fluorinated or partially fluorinated | Alkane hydrocarbons or |
| hydrocarbons/esters or ethers | —CH₂—CH₂—OH |
| (This invention) | (This invention) |

TABLE 10
Possible Donor Structures

R₃
CH₃—O—
CH₃—S—

TABLE 11
Possible Donor Structures with Prior Art Bridges and Acceptors

R₄

Some of these groups may be used to attach chromophores to the polymer backbone as a sidechain, either by first reacting the donor component onto the polymer, then completing the chromophore synthesis, or by reacting the complete chromophore onto the polymer.

It should be noted that fluorine substitution in close proximity to the donor nitrogen atom can reduce the overall efficiency (lowers λₘₐₓ and/or the dipole moment) of the chromophore.

Thus one has to carefully balance the amount of fluorine and the placement of the fluorine groups in order to synthesize chromophores that have good efficiency (high λₘₐₓ values and large dipole moments) and solubility in fluoropolymers as well as lower refractive index values for use in various waveguide applications.

TABLE 12
Effects of Donor Fluorination on Chromophore Properties

| | | | λₘₐₓ | | Solubility in | Refractive |
|---|---|---|---|---|---|---|
| | MW | μ | (nm) | FOM | fluoropolymers | index values |
| | 223.3 | 12.9 | 490 | 4.4 | Poor | High |
| | 251.3 | 13.0 | 503 | 4.4 | Poor | High |
| | 267.3 | 12.7 | 490 | 3.7 | Poor | High |
| | 309.4 | 12.9 | 486 | 3.1 | Poor | High |
| | 663.4 | 12.3 | 493 | 1.4 | Good | Lower |
| | 453.5 | 8.7 | 487 | 1.4 | Intermediate | Lower |
| | 305.3 | 11.58 | 475 (estimate) | 2.60 | Intermediate | Lower |

### Example 2

### Crosslinkable (Side Chain, Dendritic) Chromophores

In this example, we show the preferred structure of one class of the chromophores of this invention. The general structure of these chromophores is shown below, where B is a π-conjugated or aromatic bridge, and A is a π-acceptor. If DC₁ or DC₂ contain one or more fluorine atoms, then A and B can optionally contain fluorine atoms. If neither DC₁ nor DC₂ contain fluorine, then at least one of A or B must contain fluorine atoms or groups

DC=Donor-connecting link to a polymer backbone
BC=Bridge-connecting link to a polymer backbone
AC=Acceptor-connecting link to a polymer backbone

DC₁, DC₂, BC, AC each are independently selected from the following:

DC, B, BC, A, AC can each independently be pure hydrocarbon or may contain fluorine atoms as long as at least one does contain fluorine atoms. At least one of the four groups, DC₁, DC₂, AC, BC must be present for this example. If AC and/or BC are present, then DC₁ and DC₂ may also be represented by any of the R₁ and R₂ groups presented in Example 1.

The polymer will contain one or more complementary linking sites on its side chains.

The polymer must have the functional groups complementary to the functional groups on the chromophore. Optionally, additional functional groups may be present on the polymer.

The polymer can be designed so the refractive index after inclusion of the chromophore as the side chain is either low (n<˜1.49) or high (n>˜1.49).

### Example 3

### Chromophores with Novel Acceptors

In this example, we show the preferred structure of one class of the chromophores of this invention. The general structures of these chromophores are shown below, where D is a π-conjugated or aromatic donor, and B is a π-conjugated or aromatic bridge.

TABLE 13
Examples of Novel Acceptor Structures

X₁₃

TABLE 14
Examples of Novel Acceptor Structures

X₁₄

### Example 4

### Chromophores with Novel Acceptors

In this example, we show the preferred structure of one class of chromophores of this invention. The general structure of this chromophore is shown below, where D is a π-conjugated or aromatic donor, and B is a π-conjugated or aromatic bridge.

TABLE 15
Examples of Novel Acceptor Structures

| X₁₅ | X₁₆ |
|---|---|
| —CF₃ | —CF₃ |
| —CF₃ | —CH₃ |
| —CF₃ | |
| | —CH₃ |
| | |
| | |
| | |
| | —CH₃ |
| | —CH₃ |
| | —CF₃ |
| | |
| | |

### Example 5

### Chromophores with Novel Acceptors

In this example, we show the preferred structure of one class of the chromophores of this invention. The general structure of this chromophore is shown below, where D is a π-conjugated or aromatic donor, and B is a π-conjugated or aromatic bridge.

### Example 6

### Chromophores with Novel Acceptors

In this example, we show the preferred structure of one class of the chromophores of this invention. The general structure of this chromophore is shown below, where D is a π-conjugated or aromatic donor, and B is a π-conjugated or aromatic bridge.

In this example, X_{A} is a hydrocarbon alkane, which may optionally include fluorine, X_{A}=—CH₂—CₙFₘH₂ₙ₊₁₋ₘ, where n=1 to 5 and m=0 to 2n+1.

### Example 7

### Chromophores with Novel Acceptors

In this example, we show the preferred structure of one class of the chromophores of this invention. The general structure of this chromophore is shown below, where D is a π-conjugated or aromatic donor, and B is a π-conjugated or aromatic bridge.

TABLE 16
Examples of Novel Acceptor Structures

X₁₇
—CH₃
—CF₃

### Example 8

### Chromophores with Novel Acceptors

In this example, we show the preferred structure of one class of the chromophores of this invention. The general structure of this chromophore is shown below, where D is a π-conjugated or aromatic donor, and B is a π-conjugated or aromatic bridge.

TABLE 17
Examples of Novel Acceptor Structures

X₁₈
—CH₃
—CF₃

### Example 9

### Chromophores with Novel Acceptors

In this example, we show the preferred structure of one class of the chromophores of this invention. The general structure of these chromophore is shown below, where D is a π-conjugated or aromatic donor, and B is a π-conjugated or aromatic bridge.

TABLE 18
Novel Acceptor Structures

| X₁₉ | X₂₀ | X₂₁ |
|---|---|---|
| —CF₃ | —CF₃ | |
| —CF₃ | | |
| | | |

The following examples illustrate synthesis of specific chromophores based on the previous examples. The syntheses were performed as outlined previously.

### Example 10

### Chromophore with Novel Acceptors or Bridge Structures

Where R1, R2, and X₇ are described elsewhere, and where Z is an aliphatic, or partially or fully halogenated aliphatic.

### Example 11

### Synthesis of a Prior Art Chromophore (EC5)

The measured absorption maximum was 583 nm, the molecular weight was 400.48, and the dipole was calculated as 18.7 Debye, giving an FOM of 6.7. This chromophore was incorporated into Polymer A at 8.48 wt % (153-105-06), with a refractive index of 1.452. The electrooptic coefficient of the material was measured to be 5.73 pm/V at 50 V/μ poling field.

### Example 12

### Synthesis of a Prior Art Chromophore (VC1)

The measured absorption maximum was 490 nm, the molecular weight was 223.27, and the dipole was calculated as 12.86 Debye, giving an FOM of 4.41. This chromophore was incorporated into Polymer B at 4.5 wt % (153-005-11), which represented its solubility limit. The resulting film quality was determined to be 3, with a refractive index of ˜1.46. The electrooptic coefficient of the material was measured to be 2.31 pm/V at 50 V/μ poling field.

### Example 13

### Synthesis of a Prior Art Chromophore (EC10)

The measured absorption maximum was 519 nm, the molecular weight was 249.31, and the dipole was calculated as 14.75 Debye, giving an FOM of 5.58. This chromophore was incorporated into Polymer A at 5.83 wt %, (153-012-23) which represented its solubility limit. The resulting film quality was of acceptable quality, with a refractive index of 1.4654. The electrooptic coefficient of the material was measured to be 3.90 pm/V at 50 V/μ poling field.

### Example 14

### Synthesis of a Chromophore with a Novel Donor (EC12)

The measured absorption maximum was 555 nm, the molecular weight was 539.52, and the dipole was calculated as 9.32 Debye, giving an FOM of 2.07. This chromophore was incorporated into a 60/40 copolymer of methyl methacrylate and heptafluoroisopropyl methacrylate (n=1.4197) at 9.1 wt % (133-142-31). The resulting film quality was determined to be 3, with a refractive index of 1.4459.

### Example 15

### Synthesis of a Chromophore with a Novel Donor (EC32)

The measured absorption maximum was 521 nm, the molecular weight was 359.4, and the dipole was calculated as 14.08 Debye, giving an FOM of 3.74. This chromophore was incorporated into Polymer A at 11 wt % (153-044-34). The resulting film quality was determined to be 3, with a refractive index of 1.4646. Comparison to the results of Example 13, a prior art chromophore with conventional donor, shows the partially fluorinated donor has only slightly impacted both the chromophore absorption maximum and dipole moment, while greatly increasing its solubility in the test polymer.

### Example 16

### Synthesis of a Chromophore with a Novel Donor (CC1)

The measured absorption maximum was 548 nm, the molecular weight was 385.43, and the dipole was calculated as 14.43 Debye, giving an FOM of 4.29. This chromophore was incorporated into Polymer C at 12.23 wt % (153-065-23). The resulting film quality was determined to be 7, which precluded measurements of the refractive index or EO response.

### Example 17

### Chromophore with a Novel Donor (EC41-mod)

The estimated absorption maximum was 585 nm, the molecular weight was 437.49, and the dipole was calculated as 10.6 Debye, giving an estimated FOM of 3.5.

### Example 18

### Synthesis of a Chromophore with a Novel Donor (EC41)

The measured absorption maximum was 586.5 nm, the molecular weight was 383.52, and the dipole was calculated as 12.87 Debye, giving an FOM of 4.92. This chromophore was incorporated into Polymer A at 10.36 wt % (153-099-30). The resulting film quality was determined to be 5−.

### Example 19

### Synthesis of a Chromophore with a Novel Acceptor (CNC2—F5)

The measured absorption maximum was 651 nm, the molecular weight was 569.49, and the dipole was calculated as 13.24 Debye, giving an FOM of 5.0. This chromophore was incorporated into Polymer A at 10.85 wt % (133-115-23). The resulting film quality was determined to be 2, with a refractive index of 1.4524. This chromophore was incorporated into Polymer B at 9.25 wt % (133-137-29), with a refractive index of 1.4591. The electrooptic coefficient of the material was measured to be 2.73 pm/V at 50 V/μ poling field.

### Example 20

### Synthesis of a Chromophore with a Novel Acceptor (EC35)

The measured absorption maximum was 679 nm, the molecular weight was 511.45, and the dipole was calculated as 15.16 Debye, giving an FOM of 7.37. This chromophore was incorporated into Polymer A at 9.75 wt % (153-074-02). The resulting film quality was determined to be 3, with a refractive index of 1.4524. This chromophore was incorporated into Polymer A at 10.0 wt % (153-074-25). The electrooptic coefficient of the material was measured to be 5.12 pm/V at 50 V/μ poling field.

### Example 21

### Synthesis of a Chromophore with a Novel Acceptor (EC9)

The measured absorption maximum was 535 nm, the molecular weight was 365.27, and the dipole was calculated as 12.99 Debye, giving an FOM of 3.74. This chromophore was incorporated into Polymer A at 12 wt % (153-009-22). The resulting film quality was determined to be 3+. This chromophore was incorporated into Polymer A at 12.13 wt % (153-018-07) with a refractive index of 1.454. The electrooptic coefficient of the material was measured to be 6.14 pm/V at 50 V/μ poling field.

### Example 22

### Synthesis of a Chromophore with a Novel Acceptor (VC5)

The measured absorption maximum was 490 nm, the molecular weight was 373.37, and the dipole was calculated as 8.52 Debye, giving an FOM of 3.98. This chromophore was incorporated into Polymer C at 14.32 wt % (133-115-35). The resulting film quality was determined to be 5.

### Example 23

### Synthesis of a Chromophore with a Novel Acceptor (VC10)

The measured absorption maximum was 584 nm, the molecular weight was 469.37, and the dipole was calculated as 15.37 Debye, giving an FOM of 4.73. This chromophore was incorporated into a 50/50 copolymer of methyl methacrylate and heptafluoroisopropyl methacrylate at 14.05 wt % (153-069-21). The resulting film quality was determined to be 4.

### Example 24

### Synthesis of a Chromophore with a Novel Acceptor (EC23)

The measured absorption maximum was 527.5 nm, the molecular weight was 295.34, and the dipole was calculated as 11.16 Debye, giving an FOM of 3.78. This chromophore was incorporated into Polymer A at 12.48 wt % (153-020-32). The resulting film quality was determined to be 2, with a refractive index of 1.4638. The electrooptic coefficient of the material was measured to be 3.37 pm/V at 50V/μ poling field. This chromophore was incorporated into Polymer A at 11.34 wt % (153-090-10) with a refractive index of 1.4627. The electrooptic coefficient of the material was measured to be 5.88 μm/V at 50 V/μ poling field.

### Example 25

### Synthesis of a Chromophore with a Novel Acceptor (EC27)

The measured absorption maximum was 538 nm, the molecular weight was 349.31, and the dipole was calculated as 12.88 Debye, giving an FOM of 3.96. This chromophore was incorporated into Polymer A at 9.93 wt % (153-034-16). The resulting film quality was determined to be 3. This chromophore was incorporated into Polymer A at 15.0 wt % (153-049-02), with a refractive index of 1.4655. The electrooptic coefficient of the material was measured to be 4.41 pm/V at 50 V/μ poling field.

### Example 26

### Synthesis of a Chromophore with a Novel Acceptor (EC31)

The measured absorption maximum was 585 nm, the molecular weight was 431.52, and the dipole was calculated as 15.36 Debye, giving an FOM of 5.17. This chromophore was incorporated into Polymer A at 8.62 wt % (153-069-06). The resulting film quality was determined to be 8−.

### Example 27

### Synthesis of a Chromophore with a Novel Acceptor (EC42)

The measured absorption maximum was 535 nm, the molecular weight was 363.34, and the dipole was calculated as 12.44 Debye, giving an FOM of 3.60. This chromophore was incorporated into Polymer A at 12.0 wt % (153-110-02). The resulting film quality was determined to be 3, with a refractive index of 1.4568. The electrooptic coefficient of the material was measured to be 5.09 pm/V at 50 V/μ poling field.

### Example 28

### Synthesis of a Chromophore with a Novel Acceptor (VC9)

The measured absorption maximum was 600 nm, the molecular weight was 350.43, and the dipole was calculated as 16.5 Debye, giving an FOM of 7.49. This chromophore was incorporated into Polymer D at 7.78 wt % (153-067-26), with a refractive index of 1.4816. The electrooptic coefficient of the material was measured to be 3.76 pm/V at 50 V/μ poling field.

### Example 29

### Synthesis of a Chromophore with a Novel Acceptor (VC12)

The measured absorption maximum was 631 nm, the molecular weight was 446.48, and the dipole was calculated as 16.59 Debye, giving an FOM of 7.09. This chromophore was incorporated into Polymer A at 9.88 wt % (153-079-18), with a refractive index of 1.4489. The electrooptic coefficient of the material was measured to be 1.55 pm/V at 50 V/μ poling field.

### Example 30

### Synthesis of a Chromophore with a Novel Acceptor (VC13)

The measured absorption maximum was 636 nm, the molecular weight was 452.58, and the dipole was calculated as 17.15 Debye, giving an FOM of 7.44. This chromophore was incorporated into Polymer A at 10.54 wt % (153-105-18). The resulting film quality was determined to be 3−, with a refractive index of 1.4536. The electrooptic coefficient of the material was measured to be 3.74 pm/V at 50 V/μ poling field.

### Example 31

### Synthesis of a Chromophore with a Novel Acceptor (VC28)

The measured absorption maximum was 627 nm, the molecular weight was 707.00, and the dipole was calculated as 14.86 Debye, giving an FOM of 3.92. This chromophore was incorporated into Polymer A at 10.25 wt % (153-143-10). The resulting film quality was determined to be 5, with a refractive index of 1.4494. The electrooptic coefficient of the material was measured to be 3.20 pm/V at 50 V/μ poling field. The use of the massive donor groups did little to enhance chromophore performance or solubility in the low refractive index polymers.

### Example 32

### Synthesis of a Chromophore with a Novel Acceptor (EC48)

The measured absorption maximum was 654 nm, the molecular weight was 563.61, and the dipole was calculated as 17.88 Debye, giving an FOM of 6.89. This chromophore was incorporated into Polymer A at 9.19 wt % (153-150-26). The resulting film quality was determined to be 7. This chromophore was incorporated into a 50/50 copolymer of methyl methacrylate and trifluoroethyl methacrylate at 9.03 wt % (153-150-29). The resulting film quality was determined to be 3−.

### Example 33

### Synthesis of a Chromophore with a Novel Acceptor (VC8)

The measured absorption maximum was 625 nm, the molecular weight was 469.46, and the dipole was calculated as 16.9 Debye, giving an FOM of 6.64. This chromophore was incorporated into Polymer A at 9.13 wt % (153-143-26). The resulting film quality was determined to be 3−, with a refractive index of 1.4693. The electrooptic coefficient of the material was measured to be 9.83 pm/V at 50 V/μ poling field.

### Example 34

### Synthesis of a Chromophore with a Novel Acceptor (VC15)

The measured absorption maximum was 583 nm, the molecular weight was 435.42, and the dipole was calculated as 14.41 Debye, giving an FOM of 4.75. This chromophore was incorporated into Polymer A at 11.03 wt % (153-099-02). The resulting film quality was determined to be 4−.

### Example 35

### Synthesis of a Chromophore with a Novel Acceptor (VC20)

The measured absorption maximum was 582.5 nm, the molecular weight was 459.42, and the dipole was calculated as 15.94 Debye, giving an FOM of 4.96. This chromophore was incorporated into Polymer A at 11.33 wt % (153-127-31): The resulting film quality was determined to be 3−.

### Example 36

### EO Chromophores in Highly Fluorinated, Low Refractive Index, Polymer

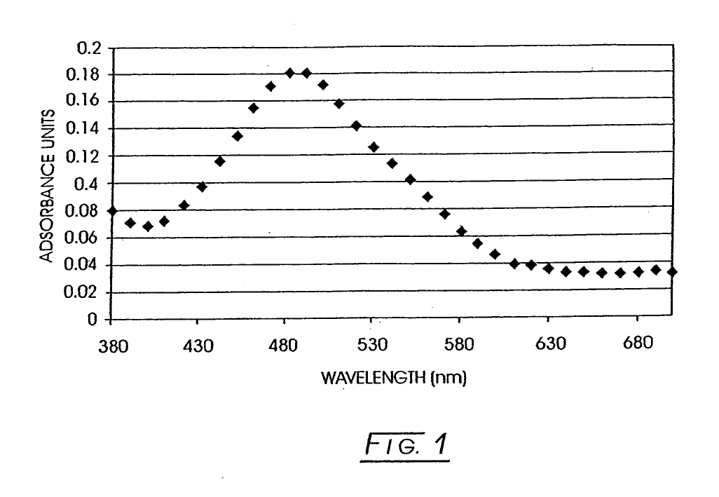

Three commercial, highly fluorinated polymers, Teflon AF (Dupont), CYTOP (Asahi Glass), and Lumiflon (Asahi Glass) were exposed to hot chromophore vapor (EC9, Example 21) over a 24-hour period. FIG. 1 shows the absorption spectrum for CYTOP stained by the chromophore. The location of the absorption maximum, and the shape of the absorption curve are similar to that of the chromophore in solution. Similar results were observed using Teflon AF and Lumiflon.

CYTOP is soluble in perfluoro-t-butyl amine. The chromophore of Example 14 (EC12) was also somewhat soluble in this solvent. A saturated solution of the chromophore in perfluoro-t-butyl amine containing 9% by weight CYTOP was filtered through glass wool and a small amount was applied to an EO test cell then dried for 2 hours on a 70 C hot plate. A measurable EO response was obtained.

### Example 37

### Sol-Gel Waveguides

Optical quality waveguides were obtained (Japan Synthetic Rubber) or made according to the recipes and processes described in “Sol-Gel Technologies for Thin Films, Fibers, Preforms, Electronics, and Specialty Shapes”, Lisa C. Klein ed., Noyes Publications, Park Ridge, N.J., 1988.

The starting refractive index of the sol-gel waveguide was 1.4752. A 10% by weight solution of the chromophore of Example 24 (EC23) in dioxane (red colored solution) was placed on the surface of the sol-gel waveguide and allowed to soak into the waveguide over a 24 hour time period at room temperature. The sample was covered and a dioxane vapor was maintained over the sample for the entire period.

After 24 hours the sample was removed from the container and the remaining solvent/chromophore was wiped off the surface of the waveguide. A refractive index measurement was then made of the now red color-stained sol-gel waveguide. The waveguide no longer exhibited a single refractive index, but instead exhibited the behavior typical of diffusion-formed waveguides, where the refractive index varies due to a concentration gradient of the dopant.

These results show how one can infuse EO chromophores into sol-gel waveguides so that electrooptically active devices can be manufactured.

### Example 38

### Synthesis of a Chromophore with a Spatial Donor Structure (CC04)

The chromophore was synthesized according to the following procedure. The diamine (CH₃CH₂—NH—CH₂C₈H₁₆CH₂—NH—CH₂CH₃) was prepared by reacting N-ethylaniline (Aldrich Chemical Company) with 1-iodibromodecane (Aldrich Chemical Company) to produce an aromatic linear spatial donor structure. Bromination of the aromatic groups followed by reaction with bridge and acceptor groups produced the final product

The measured absorption maximum was similar to that of the monomeric chromophore (Example 23). The chromophore formed poor films in low index fluorinated polymers, due to the extended hydrocarbon spacer.

### Example 39

### Synthesis of a Chromophore with a Novel Acceptor (EC38)

The measured absorption maximum was 564 nm, the molecular weight was 298.34, and the dipole was calculated as 11.1 Debye, giving an FOM of 4.73. This chromophore was incorporated into Polymer A at 9.96 wt % (178-52-30). The resulting film quality was determined to be 3− with a refractive index of 1.4538. The electrooptic coefficient of the material was measured to be 5.47 pm/V at 50 V/μ poling field.

### Example 40

### Synthesis of a Chromophore with a Novel Acceptor (EC47)

The measured absorption maximum was 559 nm, the molecular weight was 420.38, and the dipole was calculated as 12.97 Debye, giving an FOM of 3.80. This chromophore was incorporated into Polymer A at 10.23 wt % (153-150-10). The resulting film quality was determined to be 3 with a refractive index of 1.4578. The electrooptic coefficient of the material was measured to be 5.05 pm/V at 50 V/μ poling field.

### Example 41

### Synthesis of a Chromophore with a Novel Acceptor (EC50)

The absorption maximum was estimated as 650 nm, the molecular weight was 537.63, and the dipole was calculated as 17.32 Debye, giving an FOM of 6.8. This chromophore was incorporated into Polymer A at 10.33 wt % (178-17-02). The resulting film quality was determined to be 3− with a refractive index of 1.4516. The electrooptic coefficient of the material was measured to be 2.59 pm/V at 50 V/μ poling field.

### Example 42

### Chromophore with a Novel Donor (EC53-mod)

The measured absorption maximum was estimated as 640 nm, the molecular weight was 459.58, and the dipole was calculated as 14.78 Debye, giving an FOM of 6.5.

### Example 43

### Synthesis of a Chromophore with a Novel Acceptor (EC56)

The measured absorption maximum was 465.5 nm, the molecular weight was 453.38, and the dipole was calculated as 10.47 Debye, giving an FOM of 1.47.

### Example 44

### Synthesis of a Chromophore with a Novel Donor (VC3)

The measured absorption maximum was 487 nm, the molecular weight was 453.45, and the dipole was calculated as 8.68 Debye, giving an FOM of 1.43. This chromophore was incorporated into Polymer E at 9.93 wt % (133-145-17). The resulting film quality was determined to be 3 with a refractive index of 1.4637. The electrooptic coefficient of the material was measured to be 1.90 pm/V at 50 V/μ poling field.

### Example 45

### Synthesis of a Prior Art Chromophore (VC22)

The measured absorption maximum was 530 nm, the molecular weight was 250.31, and the dipole was calculated as 12.27 Debye, giving an FOM of 4.98. This chromophore was incorporated into Polymer A at 11.47 wt % (153-127-27). The resulting film quality was determined to be 6.

### Example 46

### Chromophore with a Novel Acceptor (VC33-mod)

The measured absorption maximum was estimated at 565 nm, the molecular weight was 403.38, and the dipole was calculated as 10.49 Debye, giving an FOM of 3.33.

### Example 47

### Synthesis of a Chromophore with a Novel Donor (VC35)

The measured absorption maximum was 515.5 nm, the molecular weight was 585.39, and the dipole was calculated as 11.83 Debye, giving an FOM of 1.86. This chromophore was incorporated into Polymer A at 15.82 wt % (178-52-26). The resulting film quality was determined to be 3− with a refractive index of 1.4423. The electrooptic coefficient of the material was measured to be 2.84 pm/V at 50 V/μ poling field.

### Example 48

### Synthesis of a Chromophore with a Novel Donor and Acceptor (VC36)

The measured absorption maximum was 570 nm, the molecular weight was 453.46, and the dipole was calculated as 12.04 Debye, giving an FOM of 3.51. This chromophore was incorporated into Polymer F at 8.4 wt % (178-56-02). The resulting film quality was determined to be 3 with a refractive index of 1.4602. The electrooptic coefficient of the material was measured to be 3.68 pm/V at 50 V/μ poling field.

### Example 49

### Synthesis of a Chromophore with a Novel Acceptor (VC40)

The measured absorption maximum was 530.5 nm, the molecular weight was 369.46, and the dipole was calculated as 11.38 Debye, giving an FOM of 3.14.

### Example 50

### Synthesis of a Chromophore with a Novel Acceptor (VC41-mod)

The estimated absorption maximum was 590 nm, the molecular weight was 513.46, and the dipole was calculated as 14.75 Debye, giving an FOM of 4.30.

### Example 51

### Synthesis of a Chromophore with a Novel Acceptor (VC42)

The measured absorption maximum was 561.5 nm, the molecular weight was 408.33, and the dipole was calculated as 11.81 Debye, giving an FOM of 3.62. This chromophore was incorporated into Polymer H at 9.65 wt % (178-61-26). The resulting film quality was determined to be 4− with a refractive index of 1.4675. The electrooptic coefficient of the material was measured to be 2.28 pm/V at 50 V/μ poling field.

### Example 52

### Synthesis of a Chromophore with a Novel Acceptor (VC44)

The measured absorption maximum was 483.5 nm, the molecular weight was 325.33, and the dipole was calculated as 12.85 Debye, giving an FOM of 2.88. This chromophore was found to be soluble in highly fluorinated commercial polymers with some carboxylic acid functionality (178-079-08).

### Example 53

### Synthesis of a Chromophore with a Novel Acceptor (VC46)

The measured absorption maximum was 494 nm, the molecular weight was 469.40, and the dipole was calculated as 12.95 Debye, giving an FOM of 2.18. This chromophore was found to be soluble in highly fluorinated commercial polymers with some carboxylic acid ester functionality (178-091-28). This chromophore was incorporated into Polymer I at 15.13 wt % (178-120-22). The resulting film had a refractive index of 1.45. The electrooptic coefficient of the material was measured to be 3.78 pm/V at 50 V/μ poling field.

### Example 54

### Synthesis of a Chromophore with a Novel Acceptor (VC48)

The measured absorption maximum was 545 nm, the molecular weight was 493.34, and the dipole was calculated as 13.74 Debye, giving an FOM of 3.13. This chromophore was found to be soluble in highly fluorinated commercial polymers with some carboxylic acid ester functionality (178-097-30).

### Example 55

### Synthesis of a Chromophore with a Novel Acceptor (VC49)

The measured absorption maximum was 475.5 nm, the molecular weight was 457.39, and the dipole was calculated as 6.03 Debye, giving an FOM of 0.91. This chromophore was found to be soluble in highly fluorinated commercial polymers with some carboxylic acid functionality (178-094-17).

### Example 56

### Synthesis of a Chromophore with a Novel Acceptor (EC36)

The measured absorption maximum was 643 nm, the molecular weight was 713.45, and the dipole was calculated as 17.93 Debye, giving an FOM of 5.13.

### Example 57

### Comparison of U.S. Pat. No. 5,256,784, U.S. Pat. No. 5,670,090, and This Invention

U.S. Pat. No. 5,256,784 and U.S. Pat. No. 5,670,090 teach that barbituric and thiobarbituric acids can have the range of structures shown below

TABLE 19
Prior Art Barbituric/Thiobarbituric Acid Structures

| X₈ | Y₈ |
|---|---|
| —CH₃ | —CH₃ |
| —H | —H |
| | —CH₂CH₂OH |
| | —CH₂CH₂OH |
| | —CH₂CH₂OH |
| | —CH₂CH₂OH |
| X₈ = substituted phenyl, fluorophenyl, | Y₈ = hydrocarbon alkyl or aromatic |
| or stilbenyl groups where the | groups (1-10 carbons) with or |
| substituents can be electron | without active hydrogens, or the |
| withdrawing | same as R₁ |
| X₈ = H, alkyl, aryl, | Y₈ = X₈ (all hydrocarbon) |
| CH₂(CH₂)ₓOH(SH), (all hydrocarbon) | |

The prior art barbituric/thiobarbituric acid structures are acceptors and react with the donor-bridge intermediate materials to form NLO chromophores

All the NLO compounds derived using barbituric or thiobarbituric acid acceptors disclosed or anticipated/taught in these prior patents are not compatible with the fluoropolymers used in this invention. All these prior art NLO compounds, based on barbituric or thiobarbituric acid acceptors, have high melting points, tend to crystallize out of our polymer systems, and have high refractive index values. All of these properties detract from the ability to create total low refractive index systems (polymer plus NLO chromophore) from these NLO compounds that are compatible with standard silica waveguides, presently used as platforms by the telecommunications industry.

Our invention is based on the creation of completely new barbituric or thiobarbituric acid compounds that have unique combinations of lower melting points and lower refractive indices than the prior art. The current prior art NLO chromophores based on conventional barbituric or thiobarbituric acid structures are either not soluble or not stable/compatible with the fluoropolymers described in this invention. This invention describes new barbituric or thiobarbituric acid derivatives that can be used to create new NLO chromophores with lower melting points, lower refractive indices, and are more stable/soluble in the fluoropolymers described in this invention.

### Control of Melting Points of Barbituric and Thiobarbituric Acids

Barbituric acids and thiobarbituric acids are easily derived from substituted urea or thiourea compounds

It can be shown that the melting points of the thiourea and urea starting materials greatly influence the melting points of the final barbituric or thiobarbituric acid compounds. Tables 20 and 21 contain the melting points and molecular weight information for a number of commercially available urea and thiourea compounds which are used to prepare prior art barbituric and thiobarbituric acid structures. Also Tables 20 and 21 contain two “McGinniss Equation” parameters X_{N} (weight fraction of nitrogen in the urea or thiourea starting materials) and Z′ (π electron density fraction) which are used to correlate the melting points of the starting urea or thiourea with their chemical structures (reference). In both cases there is a good correlation between the melting points, the molecular weights and the “McGinniss Equation” variables.

There is also a direct correlation between the melting points of the substituted ureas and thioureas with the melting points of their associated barbituric or thiobarbituric acid compounds (Table 22). The melting point/chemical structure correlation results shown in Tables 20-22 are applicable to a wide variety of relatively simple prior art materials but do not hold for fluorinated or nitro derivatives of urea and thiourea compounds. Table 23 gives a listing of a variety of fluorinated or nitro-substituted phenyl urea and thiourea compounds along with their melting points and associated “McGinniss Equation” variables. There is no obvious correlation of the melting points of these compounds with their chemical structure. In general one can only make the following observations:
A nitro group on a phenyl ring of a phenyl urea/thiourea may or may not raise the melting point of the final nitrated product (urea, thiourea, barbituric/thiobarbituric acid).
A fluorine group on a phenyl ring of a phenyl urea/thiourea may or may not raise the melting point of the final fluorinated product (urea, thiourea, barbituric/thiobarbituric acid).

The control of the melting points of the barbituric (BBA) or thiobarbituric (TBBA) acid derivatives is critical for the overall design of the NLO chromophores that go into the fluoropolymer systems of this invention. The NLO chromophores of interest are generally made as shown previously.

TABLE 20
Urea Barbituric Acid Starting Materials

| Urea Molecules | | | | |
|---|---|---|---|---|
| (linear alkyl and | Molecular | Melting Point | | |
| aromatic) | Weight | (° C.) | X_{N} | Z′ |
| Urea | 60.06 | 134 C. | 0.47 | 0 |
| Diethyl urea | 116.16 | 112 C. | 0.24 | 0 |
| Ethyl urea | 88.11 | 95 C. | 0.32 | 0 |
| Phenyl urea | 136.18 | 146 C. | 0.21 | 0.044 |
| Diphenyl urea | 212.25 | 239 C. | 0.12 | 0.057 |
| Butyl urea | 116.16 | 97 C. | 0.24 | 0 |
| Propyl urea | 102 | 105 C. | 0.27 | 0 |
| Dipropyl urea | 142.22 | 105 C. | 0.27 | 0 |
| Dimethyl urea | 88.11 | 102 C. | 0.32 | 0 |
| Dioctadecyl urea | 565.03 | 111 C. | 0.05 | 0 |
| Benzyl urea | 156.18 | 150 C. | 0.19 | 0.04 |
| Methyl urea | 74.08 | 97 C. | 0.38 | 0 |

Melting Point = 0.086*(Molecular Weight) + 106.224*(X_{N}) + 1878*(Z′) + 61.76
Correlation coefficient R = 0.89

TABLE 21
Thiourea Thiobarbituric Acid Starting Materials

| Urea Molecules (linear | Molecular | Melting Point | | |
|---|---|---|---|---|
| alkyl and aromatic) | Weight | (° C.) | X_{N} | Z′ |
| Thiourea | 76.12 | 177 C. | 0.39 | 0 |
| Diethyl thiourea | 132.23 | 76 C. | 0.21 | 0 |
| Ethyl thiourea | 104.17 | 112 C. | 0.27 | 0 |
| Phenyl thiourea | 152.21 | 149 C. | 0.18 | 0.34 |
| Diphenyl thiourea | 228.32 | 152 C. | 0.12 | 0.053 |
| Butyl thiourea | 132.23 | 77 C. | 0.21 | 0 |
| Propyl thiourea | 118.20 | 110 C. | 0.24 | 0 |
| Ethyl phenyl thiourea | 180.27 | 136 C. | 0.156 | 0.033 |
| Benzyl thiourea | 166.25 | 164 C. | 0.17 | 0.036 |
| Dibutyl thiourea | 188.34 | 65 C. | 0.15 | 0 |
| Methyl thiourea | 90.15 | 120 C. | 0.31 | 0 |
| 1,3 Dimethyl thiourea | 104.18 | 61 C. | 0.29 | 0 |

Melting Point = 0.336*(Molecular Weight) + 638.3*(X_{N}) + 2190*(Z′) − 100.84
Correlation coefficient R = 0.89

TABLE 22
Relationship Between the Melting Points of Urea/Thiourea Starting
Materials and Their Barbituric/Thiobarbituric Acid Counterparts

| | | | Thiobarbituric |
|---|---|---|---|
| Urea melt | Thiourea melt | Barbituric Acid (BBA) | Acid (TBBA) melt |
| point (° C.) | point (° C.) | melt point (° C.) | point (° C.) |
| Urea (134 C.) | | Barbituric acid (250 | |
| | | C.) | |
| Dimethyl | | Dimethyl barbituric | |
| urea (102 C.) | | acid (125 C.) | |
| | Thiourea | | Thiobarbituric |
| | (177 C.) | | acid (245 C.) |
| | Dibutylthiourea | | Dibutyl |
| | (65 C.) | | thiobarbituric acid |
| | | | (61-63 C.) |
| | Diethyl thiourea | | Diethyl |
| | (76 C.) | | thiobarbituric acid |
| | | | (110 C.) |
| | Dimethyl | | Diemthyl |
| | thiourea (61 C.) | | thiobarbituric acid |
| | | | (124-126 C.) |

Melting Point of Urea/Thiourea Derivatives = 0.53*(BBA/TBBA melt point) + 21.68
Correlation coefficient R = 0.89

TABLE 23
Other Thiourea/Urea Materials

| | | Melting | | |
|---|---|---|---|---|
| Fluorine/Nitro-containing | Molecular | Point | | |
| thioureas/ureas | Weight | (° C.) | X_{N} | Z′ |
| 2-fluorophenyl thiourea | 170.21 | 142 C. | 0.16 | 0.035 |
| 4-fluorophenyl thiourea | 170.21 | 163 C. | 0.16 | 0.035 |
| 3-fluorophenyl thiourea | 170.21 | 115 C. | 0.16 | 0.035 |
| 2,4-difluorophenyl thiourea | 188.20 | 165 C. | 0.15 | 0.032 |
| 2,5-difluorophenyl thiourea | 188.20 | 137 C. | 0.15 | 0.032 |
| 2,3,4-trifluorophenyl thiourea | 206.19 | 164 C. | 0.136 | 0.029 |
| 2,4,6-trifluorophenyl thiourea | 206.19 | 197 C. | 0.136 | 0.029 |
| pentafluorophenyl thiourea | 242.17 | 152 C. | 0.11 | 0.025 |
| 3-trifluoromethyl-phenyl | 220.22 | 105 C. | 0.13 | 0.027 |
| thiourea | | | | |
| 4-trifluoromethyl-phenyl | 220.22 | 144 C. | 0.13 | 0.027 |
| thiourea | | | | |
| 2-trifluoromethyl-phenyl | 220.22 | 158 C. | 0.13 | 0.027 |
| thiourea | | | | |
| 3-5-bis(trifluoromethyl)phenyl | 288.22 | 167 C. | 0.10 | 0.021 |
| thiourea | | | | |
| 4-nitrophenyl thiourea | 197.22 | 207 C. | 0.135 | 0.03 |
| 4,4′-dinitrophenyl urea | 300 | 302 C. | 0.009 | 0.04 |

No correlation between the melting points of fluorine/nitro-containing thioureas/ureas and their molecular parameters.

Thus, if the BBA or TBBA melting points are too high or too low than the overall melting point of the resultant NLO chromophore will reflect this trend. If the melting point of the resultant NLO chromophores are too high then extreme temperatures and pressures are required to apply and fill the polymer-NLO chromophore system into the fragile silica waveguide structures. High temperatures and pressures can damage the waveguides or create devices with high optical loss (Table 24). If the melting points of the NLO chromophores are too low then the polymer system could become plasticized at elevated temperatures which could lead to voltage breakdown or rapid thermal degradation of the total system (Table 24). These very practical constraints are not recognized by the prior art in that all of the prior art NLO chromophores are designed to be in all-hydrocarbon polymer waveguide structures and were not designed or will not function in a silica waveguide platform that requires a fluoropolymer as described in this invention.

TABLE 24
Prior Art Chromophores in Fluoropolymers of this Invention

| Prior art chromophore | System | Results |
|---|---|---|
| melting point of 4-nitrophenyl thiobarbituric acid is 245° C. | 6% by weight of chromophore into TFEMA₅₀/TFPMA₄₀/HEMA₁₀ terpolymer | The system was dissolved in dioxane (5% solids) and applied to an unclad silica waveguide chip. The dried film (36 hr at 70° C.) was then pressed down into the silica waveguide structure at 200° C. and 300 psi. The result was broken waveguides and no optical transmission through the chip. |
| melting point of dibutyl thiobarbituric acid is 63° C. | 6% by weight of chromophore into TFEMA₅₀/TFPMA₄₀/HEMA₁₀ terpolymer | The system processed at a lower temperature and pressure (150° C., 175 psi) but still there was very little light guiding, and under electrical breakdown testing the sample failed at 30 V/μm. |
| melting point of 4-trifluoromethyl thiobarbituric acid is 180° C. | 6% by weight of chromophore into TFEMA₅₀/TFPMA₄₀/HEMA₁₀ terpolymer | The system processed at 160-170° C. and 200 psi, but was incompatible with the fluoropolymer and had no optical transmission at 1550 nm. |
| melting point of dimethyl barbituric acid is 125° C. | 6% by weight of chromophore into TFEMA₅₀/TFPMA₄₀/HEMA₁₀ terpolymer | The system processed at 160-170° C. and 200 psi, but was incompatible with the fluoropolymer and had no optical transmission at 1550 nm. |

Control of Solubility and Refractive Index of Barbituric Acids and Thiobarbituric Acids in Fluoropolymers of this Invention

In general fluorine-containing aromatic NLO chromophore derivatives of barbituric and thiobarbituric acids have marginal solubility in the polymers of this invention. All-hydrocarbon NLO chromophore barbituric or thiobarbituric acid derivatives have almost no solubility in the polymers of this invention (Table 25). Combinations of fluorinated aromatic and fluorinated alkyl substituted barbituric/thiobarbituric acid modified NLO chromophores can be made to be soluble in the polymers of this invention (Table 26). It should be noted, however, that too much fluorine or the wrong type of fluorine substitution on the barbituric/thiobarbituric acid functionality (acceptor) of the NLO chromophore will plasticize the polymer resulting in a system that will not withstand high temperature and voltage over any extended service lifetime. High concentrations of fluorine in a molecule increase its molecular weight and sometimes a greater weight percent of the NLO chromophore is required to match the electrooptic performance of its lower molecular weight all-hydrocarbon counterpart. Higher concentrations of certain types of fluorinated NLO chromophores can plasticize the fluoropolymer which can lead to poor performance.

The synthesis method by which all the fluorine-modified barbituric/thiobarbituric acid derivatives of this invention were made are shown in Table 27.

TABLE 25
Solubility of Prior Art Chromophores in Fluoropolymers of This
Invention (Barbituric/Thiobarbituric Acid Acceptors)

| | | | Solubility in |
|---|---|---|---|
| | | | Low-Index |
| Barbituric/ | | | Polymers for |
| Thiobarbituric | | | this |
| Acid Acceptors | X₈ | Y₈ | Invention |
| Barbituric acid | H | H | Not soluble or |
| | | | compatible |
| Thiobarbituric acid | H | H | Not soluble or |
| | | | compatible |
| Dimethyl thiobarbituric | CH₃ | CH₃ | Not soluble or |
| acid | | | compatible |
| Dimethyl barbituric | CH₃ | CH₃ | Not soluble or |
| acid | | | compatible |
| Diethyl thiobarbituric | C₂H₅ | C₂H₅ | Not soluble or |
| acid | | | compatible |
| Dibutyl thiobarbituric | C₄H₉ | C₄H₉ | Not soluble or |
| acid | | | compatible |
| 4-trifluoromethyl phenyl thiobarbituric acid | H | | Not soluble or compatible |
| 4-trifluoromethyl phenyl barbituric acid | H | | Not soluble or compatible |
| 4-nitrophenyl thiobarbituric acid | H | | Not soluble or compatible |
| 4-nitrophenyl barbituric acid | H | | Not soluble or compatible |

TABLE 26
Chromophores Made From Fluorine-Containing
Barbituric/Thiobarbituric Acids Acceptors

| | | Solubility |
|---|---|---|
| | | in fluoro- |
| Fluorine-containing | | polymers |
| barbituric/thiobarbituric acid | Desig- | of this |
| acceptors | nation | invention |
| | Prior art | Not soluble (no optical clarity- high haze or high opacity) |
| | Prior art | Not soluble (no optical clarity- high haze or high opacity) |
| | This invention | Soluble and very compatible (optically clear) |
| | This invention | Soluble and very compatible (optically clear) |
| | This invention | Soluble and very compatible (optically clear) |
| | This invention | Soluble and very compatible (optically clear) |
| | This invention | Soluble and very compatible (optically clear) |

TABLE 27
Synthesis of Fluorine-Containing Barbituric or Thiobarbituric Acid
Acceptors

| X₈NCO or X₈NCS | Y₈NH₂ |
|---|---|
| CF₃(CF₂)₅CH₂NCO | CF₃CH₂NH₂ (n = 1.295) |
| CF₃CF₂CH₂NCO (n = 1.3750) | CF₃(CF₂)₆CH₂NH₂ (n = 1.365) |
| CF₃(CF₂)₂CH₂NCO (n = 1.3152) | CF₃CF₂CH₂NH₂ (n = 1.297) |
| CF₃(CF₂)₄NCO (n = 1.290) | pentafluoroaniline |
| | |
| Pentafluorophenyl isocyanate (n = 1.449) | |
| CF₃CH₂NCO (n = 1.3486) | |

It was also discovered that NLO chromophores with mixed alkane (hydrocarbon and fluorocarbon) modification of barbituric/thiobarbituric acid acceptors were also soluble in the fluoropolymers of this invention (Table 28). These mixed fluorine-alkane/hydrocarbon-alkane barbituric or thiobarbituric acid acceptors can be synthesized as follows:

TABLE 28
Solubility of Prior-Art All-Hydrocarbon Barbituric/Thiobarbituric
Chromophores and the Mixed Fluorocarbon(FC)/Hydrocarbon(HC)
Chromophores of this Invention
(Barbituric/Thiobarbituric Acid Acceptor)

| | Solubility in Low Refractive |
|---|---|
| | Index Fluoropolymers |
| Barbituric/Thiobarbituric Acid Acceptors | of This Invention |
| Dibutylthiobarbituric acid | No |
| (all hydrocarbon) | |
| X₈ = Y₈ = —(CH₂)₃—CH₃ | |
| Heptafluorobutyl, butylthiobarbituric | Yes |
| acid | |
| X₈ = —CH₂—(CF₂)₂—CF₃ | |
| Y₈ = —CH₂—(CH₂)₂—CH₃ | |
| Diethylbarbituric acid (all hydrocarbon) | No |
| X₈ = Y₈ = —CH₂—CH₃ | |
| Trifluoroethyl, ethylbarbituric acid | Yes |
| X₈ = —CH₂—CF₃ | |
| Y₈ = —CH₂—CH₃ | |

The requirement for chromophores of this invention is that they do not adversely affect the overall refractive index of the total system. This means that the chromophores should contain functionalities, such as aliphatic carbon fluorine groups, to maintain a low refractive index (n). Table 29 shows a comparison of calculated refractive index values for prior art barbituric/thiobarbituric acid acceptors and the fluorine-modified barbituric/thiobarbituric acid acceptors of this invention.

TABLE 29
Calculated Refractive Index Values for Prior Art and this
Invention's Barbituric/Thiobarbituric Acid Acceptors

| | | Refractive |
|---|---|---|
| Structure | Designation | Index |
| | Prior Art | 1.60 |
| | Prior Art | 1.70 |
| | Prior Art | 1.61 |
| | This Invention | 1.56 |
| | This Invention | 1.47 |
| | This Invention | 1.58 |
| | This Invention | 1.47 |
| | This Invention | 1.44 |

Note:
The refractive index values for all the acceptors in this table were calculated using the molar refractive index values contained in D. W. van Krevelen “Properties of Polymers” 2^{nd} Edition, Elsevier, New York, 1980 pp 212-218.

Other novel structures envisioned in this invention include the modification of the aryl group on an amine-functional acceptor to contain fluorine groups.

An additional novel modification to these acceptors is as follows:

An alternate modification is:

The following reaction scheme describes how a prior art fluorinated active methylene acceptor is synthesized

The problem with this particular fluorinated acceptor is that it is not very soluble in our fluoropolymers and the aromatic group increases the overall refractive index of the chromophore and the total system (polymer+chromophore).

New fluorinated active methylene pyrazoline-5-one compounds were synthesized as follows

Other modifications could also include reacting the phenylhydrazines with:

The contribution of the aromatic groups to the refractive index are shown in Table 30.

TABLE 30
Contribution of Aromatic Groups to Refractive Index

| | | | Contribution to |
|---|---|---|---|
| | Desig- | Solubility in | Refractive |
| Structure | nation | Fluoropolymers | Index |
| | Prior Art | 5% by weight | |
| | This Invention | 10% by weight | |
| | This Invention | 15% by weight | |

Other acceptors of this invention are as follows:

TABLE 31
Pyrazolin-5-one Acceptors

X₁₁
—(CH₃)

These acceptors can be synthesized in the following manner

J. March, Advanced Organic Chemistry, J. Wiley, New York, 1985, pp 804.

TABLE 32
5-Isoxazolone Acceptors

X₁₂

These acceptors can be synthesized in the following manner

A. Quilico, “The Chemistry of Heterocyclic Compounds”, A. Weissberger editor, vol. 17, pp 6 (1962), J. Wiley, New York.

In summary, this invention distinguishes itself from prior art barbituric/thiobarbituric acid acceptors, pyrazoline-5-one acceptors and isoxazolene acceptors in the following manner:
Melting Points—if the melting points of the acceptors are either too high (greater than 200C) or too low (less than 50C) then the resultant chromophores derived from these acceptors would not be useful in polymer systems to be processed on delicate unclad silica waveguide substrates
Solubility—if the fluorine content of the acceptor is too low (3 or fewer fluorine atoms) then the resultant chromophore will not be sufficiently soluble in a low refractive index polymer to make a total system that is compatible with silica waveguide structures.
Refractive Index—if the fluorine content is low (3 or fewer fluorine atoms) and only contained on aromatic substituents of the acceptor, then the overall contribution of the acceptor to the refractive index of the final chromophore will be high (1.5 or greater). Combinations of aliphatic fluorocarbon and fluorine-containing aromatic groups on the acceptor will lower (less than 1.5) its contribution to the overall refractive index of the chromophore.

The ability to control the refractive index of the acceptor and the final chromophore to allow a total polymer/chromophore system with refractive index in the range of 1.44-1.49, coupled with enhanced solubility of the chromophore in low refractive index polymers is vital to provide compatibility with silica optical waveguide substrates/or devices.

While the invention has been described with reference to a preferred embodiment, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In this application all units are in the metric system and all amounts and percentages are by weight, unless otherwise expressly indicated. Also, all citations referred herein are expressly incorporated herein by reference.

We claim:

1. A compound represented of one or more of the following general structures:
where M₁=O, S; M₂=O, S, and M₁=, ≠M₂.; M₃=O, CH₂; M₄=O, CH₂
where Z=H, alkane hydrocarbons, fluoroalkane hydrocarbons, fluorinated alkane esters and ethers; Z_{F}=fluoroalkane hydrocarbons, fluorinated alkane esters and ethers;
where D is one or more of the following:
wherein, M=O, S;
wherein Z=H, alkane hydrocarbons, fluoroalkane hydrocarbons, fluorinated alkane esters and ethers.
where R₁ is selected from the following
```
R₁
  (n = 0-11)
  (n = 0-11)
Partially fluorinated alkanes CₙFₘH₂ₙ₊₁₋ₘ n = 1 to 5, m = 1 to 2n − 1

CF₃—CH₂—
CF₃—CH₂—CH₂—O—CH₂—CH₂—
Alkane hydrocarbons CₙH₂ₙ₊₁ (n = 1 to 5)
Partially fluorinated hydrocarbon esters and ethers
```
```
R₂
  (n = 0-11)
  (n = 0-11)
Partially fluorinated alkanes CₙFₘH₂ₙ₊₁₋ₘ (n = 1 to 5, m = 1 to 2n − 1)

CF₃—CH₂—
CF₃—CH₂—CH₂—O—CH₂—CH₂—
Partially fluorinated hydrocarbon esters and ethers
Alkane hydrocarbons CₙH₂ₙ₊₁ (n = 1 to 5)
OH—CH₂—CH₂—
```
```
R₃
CH₃—O—
CH₃—S—


  (n = 1-4)
  R_{f} = —CH₂—CH₂—CF₃
  (n = 0-11)
  (n = 0-11)
  (n = 1-5, m = 1-5)
```
```
R₄

  (n = 1-4)
  R_{f} = —CH₂—CH₂—CF₃
  (n = 0-11)
  (n = 0-11)
  (n = 1-5, m = 1-5)
```

2. The compound of claim 1 represented by the following general structure:

3. The compound of claim 1 represented by the following general structure:

4. The compound of claim 1 represented by the following general structure:

5. A compound represented of one or more of the following general structures:
where D is one or more of the following:
where R₁ is selected from the following
```
R₁
  (n = 0-11)
  (n = 0-11)
Partially fluorinated alkane CₙFₘH₂ₙ₊₁₋ₘ n = 1 to 5, m = 1 to 2n − 1

CF₃—CH₂—
CF₃—CH₂—CH₂—O—CH₂—CH₂—
Alkane hydrocarbons CₙH₂ₙ₊₁ (n = 1 to 5)
Partially fluorinated hydrocarbon esters and ethers
```
```
R₂
  (n = 0-11)
  (n = 0-11)
Partially fluorinated alkanes CₙFₘH₂ₙ₊₁₋ₘ (n = 1 to 5, m = 1 to 2n − 1)

CF₃—CH₂—
CF₃—CH₂—CH₂—O—CH₂—CH₂—
Partially fluorinated hydrocarbon esters and ethers
Alkane hydrocarbons CₙH₂ₙ₊₁ (n = 1 to 5)
OH—CH₂—CH₂—
```
```
R₃
CH₃—O—
CH₃—S—


  (n = 1-4)
  R_{f} = —CH₂—CH₂—CF₃
  (n = 0-11)
  (n = 0-11)
  (n = 1-5, m = 1-5)
```
```
R₄

  (n = 1-4)
  R_{f} = —CH₂—CH₂—CF₃
  (n = 0-11)
  (n = 0-11)
  (n = 1-5, m = 1-5)
```

6. The compound of claim 5 represented by the following general structure:

7. The compound of claim 5 represented by the following general structure:

8. The compound of claim 5 represented by the following general structure:
where A is
—CH═CH—X₆
where X₆, is:
X₆
